(19) United States
(12) Patent Application Publication
(10) Pub. No.: US 2008/0172247 A1
(43) Pub. Date: Jul. 17, 2008

(54) Method to Decrease the Risk of a Vaccine-Induced Chronic Immune Mediated Disorder in Humans With a Family History of the Disorder

(75) Inventor: John Barthelow Classen, Baltimore, MD (US)

Correspondence Address: BROWDY AND NEIMARK, P.L.L.C.;624 NINTH STREET, NW, SUITE 300, WASHINGTON, DC, 20001-5303, US

(73) Assignee: CLASSEN IMMUNOTHERAPIES, Baltimore, MD (US)

(21) Appl. No.: 11/547,720

(22) PCT Filed: Apr. 6, 2005
(86) PCT No.: PCT/US2005/011386

§ 371 Date: Mar. 6, 2008

Related U.S. Application Data
(60) Provisional application No. 60/559,432, filed on Apr. 6, 2004.

(51) Int. Cl. G06Q 50/00; A61K 39/00; G06Q 30/00

(52) U.S. Cl. 705/2; 705/14

(57) ABSTRACT

A method of safer immunization against infectious diseases by considering the potential recipients family history or other risk factors for a chronic immune mediated disorders, especially type 1 diabetes prior to immunizing the recipient. The method may include screening potential recipients of a vaccine and selectivity avoiding immunizing or administering a lower risk immunization schedule to those at increased risk for the chronic immune mediated disorder. The resulting method will lower the risk of the recipient in developing the immune mediated disorder.

### BACKGROUND OF THE INVENTION

1. Field of the Invention

The present invention involves the fields of immunology and medicine, and more particularly relates to immunization methods, and compositions used therewith.

2. Related Background Art

The ability of vaccines to exacerbate disease in people with rheumatic diseases and multiple sclerosis has been studied many times. The opinions are varied but most authors have concluded it is safe to administer the vaccines to people with autoimmune disease. It has also been published that people with an previous vaccine adverse event are more likely to have the same adverse event following a second dose of the vaccine or a different vaccine. The effect of vaccines in patients with a family history of autoimmune disease has been studied only a few times. Graves (Graves et al., 1999) studied the effect of immunization on the development of islet cell autoimmunity in 317 children with first degree relatives. The authors concluded there was a lack of an association between early childhood immunization and beta cell autoimmunity. A German group did a similar study and came to the same conclusion (Hummel et al., 2000).

More recently a study from a Danish vaccine manufacturer (Hviid et al., 2004) studied the effect of immunization on the development of diabetes in children with a family history of type 1 diabetes. For the purposes of Hviid et al., a family history of type 1 diabetes meant that the study children had at least one sibling who had developed type 1 diabetes prior to age 14 (page 1400, col. 1). Siblings were defined as children having the same mother.

Hviid et al. declared, “Although we found that the risk of type 1 diabetes increased among children who had one or more siblings with diabetes, there was no apparent association between diabetes and vaccination among such children.” They elaborated, “The development of type 1 diabetes in genetically predisposed children (defined as those who had siblings with type 1 diabetes) was not significantly associated with vaccination.”

“However, the lack of statistical significance and inconsistency limit the conclusions that can be drawn from this analysis. If we consider the results for at least one dose of vaccine, the diphtheria, tetanus, and inactivated poliovirus vaccine and the oral poliovirus vaccine stand out, with rate ratios of 3.03 (95 percent confidence interval, 0.41 to 22.63) and 2.01 (95 percent confidence interval, 0.46 to 8.71), respectively. However, these increases are clearly based on reference groups that included only one and two unvaccinated children in the case of diphtheria, tetanus, and inactivated poliovirus vaccine and oral poliovirus vaccine, respectively.”

Hviid et al. end the paper by saying “In conclusion, there appears to be no support for any causal relation between childhood vaccination and type 1 diabetes.” The analysis used in the paper was flawed, as discussed in more detail in Example 1, and caused the authors to draw an incorrect conclusion.

In part because of that incorrect conclusion, Hviid et al. failed to recommend that those in the art consider whether immunization would further increase the risk of contracting type 1 diabetes in subjects who were already at an elevated risk of developing it because of a family history of diabetes. Hviid et al. did not change the labeling of any vaccine or recommend a change in the labeling of any vaccine to take the attributable risk associated with immunization in these high-risk patients into account. The reason being, of course, that Hviid et al. did not perceive that there was such an attributable risk to be considered.

Prior publications have provided comparisons of the risk of vaccine induced diabetes in people of different nationalities, sex and age. In all of these studies subgroups the risk factors were not substantially increased and the underlying incidence of IDDM was generally less than a 10 or even 5 or even 2 fold difference from the population in general and did not have a significant impact in immunization decision.

As example of immunization risks in certain risk groups, Classen (Classen, J. B., and Classen, D. C.: The safety of military immunization and the risk of insulin dependent diabetes. Clinical Practice of Alternative Medicine 2:247-252, 2001.) looked at the risk of vaccine induced diabetes in different ages groups of white men, white women, black men and black women in the US navy. The authors concluded, “In adults immunization should be withheld in individuals at high risk for autoimmune disease. In those with average risk for autoimmune disease the number of doses of vaccine should be limited to optimize the benefit to risk, as an dose response between vaccines and IDDM has been found with the hemophilus vaccine and the measles mumps rubella vaccine.”

The statement was a philosophical statement since it did not provide instructions or information for one to make an immunization recommendation. The paper did not provide data to quantify risk or say how risk should be estimated (by history or gene analysis) or what high risk is. It did not specify whether to use relative risk or absolute risk. It does not provide advise for specific vaccines especially those given to children. It does not provide advice in children except to imply immunization can be given starting at birth. Its advice for children is limited because a child may be born low risk, without a sibling with diabetes, however at a later time the child may become high risk, because a sibling develops diabetes. Immunization at birth is not an option since the child may be several years old at the time the child becomes high risk. No prior studies have found any data which should be provided to patients or clinicians so that one can use ones family history or medical history to individually access one's risk of vaccine induced chronic immune mediated disorder prior to immunization.

The inventor of the current invention has patented several methods to improve the safety of immunization. U.S. Pat. No. 5,728,385 claims to methods of reducing chronic immune mediated disorders by early immunization. U.S. Pat. No. 5,723,283 claims both a method of screening immunization schedules to determine if they are associated with autoimmunity and a method of immunization which includes comparing the risk of immunization prior to immunizing. U.S. Pat. Nos. 6,638,739 and 6,420,139 claim safer methods of immunization which comprise considering the risk of different immunization schedules prior to immunization. None of these methods specifically dealt with assessing the risk, especially the absolute risk, in groups already at high risk for a chronic immune mediated disorder prior to deciding on immunization. There was previously no data to allow individual risk assessment of immunization except for race, nationality, and sex, where the risk was not overwhelmingly different in the groups. The prior data had not suggested that practitioners should be warned to perform additional inquiries of a patient's risk based on history, family history or genetic screening prior to immunization. It is now known, as shown in this document that such screening should be performed and provides valuable risk assessment.

The specifications of patents '283, '385, '739, and '139 all teach that it MAY be easier to demonstrate a benefit (of particularly early immunization) in certain subgroups. In particular the specifications indicate that it may be easier to show a beneficial effect of early immunization in groups at high risk for chronic disease. They do not teach that vaccine manufacturers should provide specific warnings that a vaccine may cause a chronic immune mediated disorder more frequently in a particular high risk group, such as one with a family history of diabetes or in other groups where the risk of a chronic immune mediated disorder is at 10 or 40 fold increased over the general population. They do not provide the information necessary to consider the risk of vaccine-induced chronic immune mediated disorder in individual patients based on history.

The prior Classen patents further teach that, in the contemplated screening of potential immunization protocols, “The screening method may be designed to control for and or estimate the effects on at least one mammal in at least one treatment/control group from at least one confounding variable. For example, one may consider the effects of receiving breast feeding versus bottle feeding prior to 12 months of age, receiving antibiotics during the first 12 months of life, the maternal age at birth, the presence of a chronic immune mediated disorder in the mother/father/a close relative, maternal infections while the mammal was in utero, infections during the first 12 months of life, size of the mammal at birth, gestational age of the mammal at birth, exposure of the mammal to BCG vaccine or naturally acquired mycobacterium, and exposure to various vaccines or pathogens.”

The prior Classen Patents contemplated that certain immunization protocols not only protected against one or more infectious diseases, but could actually reduce the risk of developing diabetes or other chronic immune-mediated disorders. Hence, they contemplated either administering protective immunogens according to these preferred protocols to all those in risk of contracting the infectious disease, or just to those who were in the subpopulations having a high background risk of developing CIMD. The Classen Patents stated: “The present invention is intended to utilize low or non toxic agents that can be used to prevent disease in asymptomatic mammals without the need to screen them for risk of developing chronic immune mediated disorders, however the invention may be used in certain subpopulations at higher risk for developing the disorders than others. Alternatively the invention may be administered to a large number of mammals with different risks for developing a given chronic immune disorder and only certain subpopulations may be shown to benefit statistically from the administration. The subpopulations may include mammals at higher risk than the general population. Non-limiting examples of the subpopulations include those with family history of at least one chronic immune mediator disorder, those who are deemed at high risk because of genetic or biochemical screening of themselves or biological relatives, and those at risk because of an abnormal birth as in prematurity or small size.” [emphasis added]

The prior Classen Patents also contemplated the screening of immunization schedules to determine their effect on the risk of developing a chronic immune-mediated disorder. In this context, they noted that it might be necessary to compensate for confounding variables, such as “breast feeding, receiving antibiotics, the maternal age, family history of diabetes or a second chronic immune mediated disorder, maternal infections while the mammal was in utero, infections during the first 12 months of life, size of the mammal at birth, gestational age of the mammal at birth, and exposure to vaccines.”

The reason for compensating for confounding variables is that the difference between the two groups may not be due to immunization differences but may be due to difference other than immunization that exist in the groups. For example an immunized group may have an higher rate of diabetes than the unimmunized group because there are more people in the immunized group that have a family history of diabetes and would thus be more likely to develop diabetes.

Compensation for a confounding variable can be made without estimating or considering the risk in a subgroup. The compensation for the confounding variable can be made, for example, simply by excluding all those with the confounding variable from the comparison of the two groups. The incidence of disease in each of two groups can be calculated after removing all of those with a confounding variable.

Compensation for confounding variable does not answer the following questions: Can a person with the confounding risk factor receive the immunization safely? What is the risk of immunization to person with the confounding risk? Is the risk of immunization in the persons with the confounding risk so high that the user and immunizer should receive special warnings?

In conclusion, it has heretofore not been clearly shown or recognized that vaccines significantly increase the relative or absolute risk or attributable risk of chronic immune mediated disorders in a person already at increased risk of developing a chronic immune mediated disorder, such as a family history of such disorders, above that in a person with no increased risk. It does not teach that vaccine manufacturers should provide specific warnings about the risk that a vaccine may cause a chronic immune mediated disorder in a particular high risk group, such as one with a family history of diabetes or in other groups where the risk of a chronic immune mediated disorder is at 5, 10, 20, 30, or 40 fold increase over the general population. It does not indicate that one should separately consider the risk of immunization for each individual based on history or genetic strains. It does not provide what the absolute risk may be.

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

### SUMMARY OF THE INVENTION

The present invention relates to the discovery that persons with a family history of autoimmunity and other chronic immune mediated disorders have an increased and absolute (attributable) risk of developing such disorders following immunization.

Immunization is associated with a similar or higher relative risk of chronic immune-mediated disorders in children with a family history of such disorders compared to children that do not have a family history of such disorders.

The absolute risk of chronic immune mediated disorders is higher in children with an family history of such disorders and the absolute or attributable risk of vaccination induced chronic immune mediated disorders is also increased in children with a family history of such disorders, compared to children with no such history.

Immunization can be made safer by screening a patient's history for an increased risk of a chronic immune mediated disorder, in particular, a family history of type 1 diabetes. For example, one can ask the patient or the patient's guardian if a sibling, parent, grandparent, or cousin has or had a chronic immune mediated disorder including type 1 diabetes. A child with a family history of IDDM (three generations) has a 5 fold increased risk of developing type 1 diabetes.

The risk of developing type 1 diabetes irrespective of immunization differs depending on which relative developed diabetes. A child with a parent with type 1 diabetes may be 10 times more likely to develop diabetes than a child without a family history of diabetes. A child with a father with type 1 diabetes may be at greater risk for developing diabetes than a child with a mother with diabetes. A child with a sibling with IDDM for example may be 40 times more likely to develop diabetes, than other children without a family history of diabetes. A child with an identical twin with type 1 diabetes is at greater risk of developing diabetes than a child with a non identical twin with diabetes.

Moreover, a family history of type 2 diabetes is relevant to whether there is an increased risk of a child developing type 1 diabetes. A child with a family history of type 2 diabetes (three generations) has a 3 fold increased risk of developing type 1 diabetes.

In cases where there is a family history of diabetes (type 1 and/or type 2) a medical provider may counsel the patient or the patient's guardian about the risk of immunization and may attempt to minimize the risk of inducing diabetes by, e.g., withholding immunization, or immunizing in a manner that reduces that risk. As taught by the above-cited Classen patents, the risk of vaccine-induced immunization is dependent on the immunization schedule, and can be reduced giving vaccines starting in the first month of life, or using a decreased dosage of vaccine or fewer doses.

Safer vaccine kits can be provided by including the warnings on the package inserts.

### DETAILED DESCRIPTION OF THE PREFERRED EMBODIMENTS OF THE INVENTION

A Chronic Immune-Mediated Disorder (CIMD) is considered to be “vaccine-induced” if exposure to a vaccine is associated with an increased risk of acquiring the CIMD in the exposed group relative to the risk in the non-exposed group. It is not necessary that the vaccine be proven to be a cause (sole or contributing) of the CIMD; it is enough that it is a “risk factor”.

A subject is deemed to have a “family history” of a CIMD (Chronic Immune-Mediated Disorder) if one or more persons, related within three degrees of consanguinity, developed that CIMD. The blood relatives belonging to the first degree of consanguinity are the subject's father, mother, siblings (with at least father or mother in common), and children. The blood relatives belonging to the second degree of consanguinity are the subject's aunts and uncles, grandparents, first cousins, and grandchildren. The blood relatives belonging to the third degree of consanguinity are defined according to standard genealogical principles. It should be understood that if a relative is related to the subject by more than one chain of relationship, the shortest chain controls. Also, it should be understood that only blood relatives, not step-relatives, are considered in ascertaining family history. In considering family history, the practitioner may, if desired, limit consideration to closer relatives, e.g., those in just first degree relationships, or in just first or second degree relationships. The consideration may also be further limited, e.g., just siblings.

ABSOLUTE RISK is the probability that a member of a particular group will experience an adverse event. In the present context, the adverse events of interest are both infectious diseases which are induced by infection and which might be controlled by immunization, and chronic immune-mediated disorders which might be the result of one or more of a variety of possible causes, including immunization, infection, environmental insult, and inherited traits. The risk may be expressed as a simple rate, e.g., 1 per 100,000 persons, or as a complex rate, e.g., 1 per 100,000 persons per year.

ATTRIBUTABLE RISK measures the “excess risk” accounted for by exposure to a particular risk factor, and is thus the absolute risk in the exposed group, less the absolute risk in the non-exposed groups. It is thus a “risk difference”. If the exposure is beneficial, then the attributable risk is negative.

RELATIVE RISK (RR) is the ratio of two absolute risks, in particular, the ratio of the absolute risk in the exposed group to the absolute risk in the unexposed group. It therefore is sometimes referred to as the “risk ratio”. If the exposure is beneficial, the relative risk will be less than one.

It is possible to express these concepts mathematically by reference to a 2×2 table. However, it should be noted that the invention is not limited to the situation in which there are just two groups (treatment and control) and a single yes/no outcome.

| | Event-Yes | Event-No | Patients in Group |
|---|---|---|---|
| Treatment | A | B | (A + B) |
| Control | C | D | (C + D) |
| Total | (A + C) | (B + D) | (A + B + C + D) |

The frequency of the outcome event is (A+C)/(A+B+C+D).
The absolute risk in the treatment group is then A/(A+B), the absolute risk in the control group is C/(C+D),
the relative risk is the ratio of the former to the latter, or (A*(C+D))/(C*(A+B)), and
the absolute risk is the former, less the latter, or (A/(A+B))−(C/(C+D)).
One may also define the odds ratio, which is (A*D)/(C*B). Note that the odds ratio slightly overestimates the relative risk (the risk ratio), but that, as the frequency of the outcome event approaches zero, the odds ratio approaches the risk ratio.

### Immunization of High Risk Recipients

Manufacturers and their representatives, including those representing them in government, often deny that products produced by the manufacturer are dangerous. They typically argue that statistical associations do not prove causation. However statistical associations between a product and an adverse event provide evidence of risk unless it is already been proven that there is a known causal agent and the association between the product and the adverse event is an indirect, non causal, association. By avoiding all products that are statistically associated with an adverse event one will decrease risk of the adverse event because some of the products will likely cause the adverse event even though some of the associations are non causal associations.

Statistical significance is not needed for a risk to exist or for providing evidence of a risk. An elevated rate of an adverse event in a treatment group compared to an untreated control group indicates a risk even when the difference is not statistically significant. In many incidences the population size of the treated group and or the control group is so small that a clinically significant increased rate of an adverse event, such as death or the development of a chronic immune mediated disorder, does not reach statistical significance even when it is known the treatment causes the adverse event. Therefore risk can exist in the absence of a statistical significant difference.

The present invention pertains to the discovery that children with a family history of a chronic immune mediated disorder have an increased absolute risk, and at times also an increased relative risk, of developing the disorder, relative to the general population, and associated with exposure to immunization. The relative risks associated with immunization in children with a family history of diabetes were similar to the general population but at times significantly higher. However the absolute risk is consistently higher. For example, as described in Example 1 below, the relative risk associated with immunization with 3 doses of oral polio is 4.71 in the population with a family history of diabetes compared to 2.52 in the general population. However the attributable risk, expressed as a rate difference, presented by immunization with 3 doses of polio vaccine in the population, for subjects with a family history of diabetes was 720 cases/100,000 persons/year (rate 914-rate 194) compared to 12 cases/100,000 persons/year (rate 20.86-rate 8.27) in the general population.

The differences in attributable risk of vaccination in those with a family history versus those without a family history of diabetes are quite large.

Medical/Social history is also valuable in estimating one's risk for type 1 diabetes or other chronic immune mediated disorders. Several factors have been shown to be associated with an increased risk of chronic immune mediated disorders including type 1 diabetes. Preferably one should ascertain a history of receiving breast feeding versus bottle feeding prior to the first 12 months of age, receiving antibiotics during the first 12 months of life, the maternal age at birth, maternal infections while the patients was in utero, infections during the first 12 months of life, size of the patient at birth, gestational age at birth, exposure to BCG vaccine or naturally acquired mycobacterium, and exposure to various vaccines or pathogens, history of chronic immune mediated disorders.

The attributable risk in example 1 was determined by data from a cohort analysis. Data would be more preferably obtained from a prospective randomized clinical trial. Data could also be obtained using a case control design however in the inventor's opinion this is less preferable than a cohort study in part because case control studies are not accurate in situations where immunization of the population approaches 100% as it does with many childhood vaccines in the US. Preferably the data obtained and provided will be statistically significant.

Example 1 discloses clinically significant findings of differences in attributable risk associated with immunization, and physicians should consider this information in making their decision on whether to immunize a patient. Ideally this information should be used in conjunction with any data pertaining to the risk an individual may have for developing the infection which the vaccine protects against and the risk of serious complications from the infection. Studies may give misleading results on a vaccine's safety if one does not take into consideration that having a disease like measles may be associated with a higher incidence of a chronic immune mediated disorder than receiving the vaccine for the disease. A vaccine may induce chronic immune mediated disorders but at a lower rate than the infectious agent it protects against.

Preferably, the above mentioned risk information is included in the labeling for a kit with the vaccine. However, it is anticipated that some vaccine manufacturers or marketer/distributor/reseller may help inform physicians/healthcare providers/consumers by using other methods of disseminating this information. For example, and without limitation, a vaccine manufacturer or marketer/distributor/reseller may inform potential consumers by providing or assisting the dissemination of information electronically (e.g., over the internet), in print (e.g., academic publications, catalogues, advertisements), and orally (e.g., in presentations at conventions and at hospital or HMO staff meetings or retreats). The manufacturer or marketer/distributor/reseller can disseminate the information directly (e.g., by placing advertisements, making presentations, and distributing publications) or it can act indirectly to disseminate the information (e.g., by subsidizing a person writing a publication, subsidizing a journal where the publication is printed, or subsidizing a meeting where the information is being presented).

It is the intention of the inventor to claim safer methods of marketing a vaccine which comprise providing the newly discovered information as well as the vaccine, whether or not simultaneously, and regardless of the means used to disseminate the information.

There are many ways the discoveries can be incorporated into safer methods of immunization.

The physician can screen patients for a family history of a chronic immune mediated disorder and avoid immunizing the patients with a family history.

The physician can screen those with a family history of a chronic immune mediated disorder and immunize only those with an elevated risk of developing a serious infection which is prevented by a vaccine.

The physician can start the immunization schedule in the first month of life to reduce the risk of a chronic immune mediated disorder.

The physician can decrease the number of doses or amount of vaccine given after 42 days of life.

The physician may administer vaccine formulations less likely to induce an chronic immune mediated disorder.

A vaccine (immunogenic formulation) supplies one or more immunogens. A formulation may be simple (a recombinant immunogen in homogeneous form) or complex (a killed or attenuated pathogenic cell). The formulation may be cellular or acellular. If acellular, the immunogen may be unbound, or bound to a carrier. The immunogen may also be naturally produced, or synthetic, the latter including recombinant immunogens.

A physician may substitute a live vaccine with a killed vaccine or killed vaccine for a live vaccine. The physician may administer a vaccine without an adjuvant for example a tetanus toxoid instead of a tetanus vaccine with an aluminum adjuvant or a tetanus vaccine mixed with diphtheria toxoid. The physician may administer an acellular pertussis vaccine as compared to a whole cell pertussis vaccine. The physician may administer a diphtheria tetanus vaccine instead of a diphtheria, tetanus, pertussis vaccine.

The current invention could be modified for other high risk subjects. For example those at increase risk of chronic immune mediated disorder based on genetic screening. In this case the relative risk may be the same as the general population but the attributable or absolute risk will be higher. Alternatively, an individual with a history of one autoimmune disorder is at increased risk for an second autoimmune disorder. While the relative risk may be the same as in the general population the attributable or absolute risk will be increased.

There are a number of alterations of these methods and combinations of methods that one skilled in the art may use. It is the intention of the inventor to include these. It is not the intention of the inventor in this application/patent to claim ways of immunizing a person with an autoimmune disease such as lupus or multiple sclerosis such that the immunization does not cause an exacerbation of the disease. It is also not the intention of the author to claim the prevention of the reoccurrence of an adverse event by avoiding administering the same vaccine or a different vaccine. An example of this would be reoccurrence of vaccine induced Guillain Barre syndrome following a second immunization by avoiding future immunizations.

### Kits

The present invention also encompasses kits for administration of one or more vaccine immunogens according to the methods of the present invention. A kit typically comprises one or more receptacles, each receptacle comprising an immunogenic composition (vaccine) which in turn comprises one or more immunogens, together with directions for use and warnings concerning possible adverse effects.

The improved kits contain specific information that would lead the immunizer to inquire about the family history or medical history relevant to the risk of a chronic immune mediated disorder prior to immunizing the recipient. The information may include warning about the risk of the chronic immune mediated disorder and or include suggestions on way to avoid the risk.

The kit may contain information that the absolute risk is elevated in those with a family history. For example it may contain data on the relative risk that those with a parent with diabetes may be 10 times more likely of developing diabetes than those with no family history. It may contain data that those with a sibling may with the chronic immune mediated disorder may be 40 times more likely to develop a chronic immune mediated disorder than one with out. The Kit would also preferable have data on the absolute risk of developing such a chronic immune mediated disorder associated with receiving one or more immunogens is in the range of such as 50, 100, 150, 200, 250, 300, 350, 400, 450, 500, 550, 600, 650, 700, 750, 800, 850, 900, 950 cases or more/100,000. Preferably the kit would contain the risk of complications from the natural infection which is prevented by the vaccine. Preferably, the risk of the infection and the risk of the complications would be in the same format for easy comparison, such as in rates (e.g., cases/100,000 people).

Preferably, the kits would contain information on the risks of developing a chronic immune mediated disorder with different immunization schedules. For example the kit may provide information on a dosing effect, e.g., that the more doses of the vaccine given after 8 weeks is associated with an increased risk. The kit may provide information that starting immunization within the first month of life may be associated with a decreased risk of a chronic immune mediated disorder compared to immunization starting after 8 weeks. The kit may provide information on the risks of two different vaccines that protect against the same disease so that the health care provider or user can better choose which product to use. For example the kit may provide information on both a whole cell and non whole cell pertussis vaccine or on both a live and killed polio vaccine.

### Chronic Immune-Mediated Disorders

A chronic immune mediated disorder is one which lasts longer than two months, but does not include permanent sequela of acute immune response diseases such as seizures and anaphylaxis, nor do such disorders include diseases associated with live virus infections as in subacute sclerosing panencephalitis induced by measles vaccine. Chronic immune mediated disorders does not include acute onset sequela caused by chronic infections by live vaccines. The invention is especially useful in preventing chronic immune disorder which develop at least one year after a vaccination. Thus, an illness like Guillean-Barrea syndrome is not routinely considered an chronic immune mediated disorder.

A growing number of human diseases have been classified as autoimmune in nature (see, Theofilopoulos, A., in: D. P. Stites, et al., eds., Basic and Clinical Immunology, Lange Medical Publications, Los Altos, Calif., 1988; and Berkow, supra), which references are entirely incorporated by reference, and the present invention is intended to include as a chronic immune mediated disorder any and all of such diseases, with the exception listed herein, in mammals including humans.

Methods and compositions of the present invention may be used for protecting against and/or inhibiting chronic immune mediated disorders, which include autoimmune disorders, immune mediated cancers and hyperactive immune responses.

Autoimmune diseases include type 1 diabetes, conventional organ specific autoimmunity, autoimmune cytopenias, and autoimmune-associated neurological disease, and rheumatic diseases/connective tissue disease.

Conventional organ specific autoimmunity include thyroiditis (Graves+Hashimoto's), gastritis, adrenalitis (Addison's), ovaritis, primary biliary cirrhosis, myasthenia gravis, gonadal failure, hypoparathyroidism, alopecia, malabsorption syndrome, pernicious anemia, hepatitis, anti-receptor antibody diseases and vitiligo.

Autoimmune neurological diseases may include schizophrenia, Alzheimer's disease, depression, hypopituitarism, diabetes insipidus, sicca syndrome and multiple sclerosis.

Autoimmune rheumatic diseases/connective tissue diseases include or may include rheumatoid arthritis, systemic lupus erythematosus (SLE) or Lupus, scleroderma, polymyositis, inflammatory bowel disease, dermatomyositis, ulcerative colitis, Crohn's disease, vasculitis, psoriatic arthritis, exfoliative psoriatic dermatitis, pemphigus vulgaris, Sjorgren's syndrome.

Other diseases which can be autoimmune-related are uveoretinitis, glomerulonephritis, post myocardial infarction cardiotomy syndrome, pulmonary hemosiderosis, amyloidosis, sarcoidosis, and aphthous stomatitis, as presented herein and known in the related arts. See, e.g., Berkow et al, eds., The Merck Manual, 16th edition, Merck and Co., Rahway, N.J., 1992, pages 303-364, 710-718, 1083, 1269, 1305-1377, 1338 1677-1684, and 2435-2438 which is entirely incorporated herein by reference.

Immune mediated cancers include lymphoreticular neoplasia, lymphoblastic leukemia, brain tumors, gastric tumors, plasmacytomas, multiple myeloma, leukemia, connective tissue tumors, solid tumors and lymphomas.

Hyperactive immune responses include asthma/allergies and autoimmune diseases. Such allergies may include hay fever, atopic dermatitis, urticaria, perennial rhinitis, allergic conjunctivitis, pulmonary diseases, food allergies, skin allergies, anaphylaxis (e.g., associated upon exposure to blood products) and pollinosis.

Type I diabetes mellitus is a disease that results from deficient insulin secretion leading to hyperglycemia and ketoacidosis if the patient does not receive exogenous insulin. The destruction of the islet cells may occur gradually so patients are diagnosed with diabetes but don't require insulin initially.

There are multiple sequalae to chronic immune mediated disorders. As non-limiting examples, autoimmunity can result in end organ failure or cancer. Chronic inflammation, as occurs in chronic immune mediated disorders, can cause the release of molecules like serum amyloid which can cause pathology. Serum amyloid is associated with amyloidosis peripherally and senile dementia in the central nervous system.

### Immunization Schedule

An immunization schedule is a program for the administration of one or more specified doses of one or more specified immunogens, by one or more specified routes of administration, at one or more specified ages of the immunization subject. A supplemental immunization schedule is one intended to supplement a standard immunization schedule which is commonly followed in the region in which the subject resides.

While the immunogens which can be administered according to the present invention are discussed in detail in a later section, certain prefatory remarks regarding their place in the immunization schedule are appropriate here. The immunization schedule may provide for one or more administrations of a single immunogen, multiple immunogens which collectively immunize against the same or different strains for the same infectious disease, or multiple immunogens which collectively immunize against a plurality of different infectious diseases. The immunogens may be separately or simultaneously administered, and, in the latter case, may be combined into a single pharmaceutical composition for ease of administration.

### Immunogens

It is the intent of the inventor to limit this invention to vaccine immunogens used to prevent infectious diseases and not immune modulators. The following definitions help differentiate vaccine immunogens which are designed to prevent against specific infections versus immune modulators which are used to treat infections such as infections with viral hepatitis.

Immunogens correspond to a class of molecules that elicit an immune response through classical immunologic pathways as in the non-limiting example of the incorporation in an MHC molecule of an antigen processing cell where the immunogens can potentially interact with antigen specific T cell receptors. Alternatively, as another non limiting example, immunogens can bind to antigen specific binding regions of immunoglobulins which may lead to stimulating the B lymphocytes (if on the surface of B lymphocytes), but alternatively could elicit an immune response through other means, e.g., by the activation of complement, or the modulation of Fc receptors.

An Immunogen of the present invention is a molecule which may be derived from a virus, bacteria, yeast, mold, plant, insect, allogeneic or xenogeneic animal or compound or composition that immunologically cross reacts with a naturally occurring immunogen. Such agents may be made from the killed or live bacteria, killed or live viruses, recombinant or chemically synthesized or purified immunogenic agents including antigens, fragments or cross reacting synthetic or recombinantly produced peptides, carbohydrates, lipids or any combination thereof. Such agents can be combined with each other and with vaccines against infectious diseases to substantially prevent or reduce the incidence of immunologic disorders according to the present invention.

The term “immunologically cross reacts” refers to molecules that induce antibodies or T-cells that bind to the cross reactive molecule or fragments thereof.

Weak immunogens may be limited to the ability to invoke changes in such immune mediator cells, such as lymphocytes (B and/or T cells), macrophages and natural killer cells, such as the release of lymphokines, altered cell movement, or altered composition of cell surface receptors. Strong immunogens have the additional ability to cause either an humoral immune response (such as, e.g., antibodies to said agent) or a cellular immune response (such as, e.g., a delayed type skin reaction to said agent).

There are several examples of conventional immunogens. The classical example is that of vaccines as in human vaccines. Such vaccines may be classified as living where such agents may multiply or perform homeostatic metabolic activity in the recipient, as in the live oral polio, live BCG, and live small pox vaccines, as non-limiting examples. Alternatively, conventional vaccines can be classified as inactivated (killed), where such agents have lost their ability to multiply or maintain homeostatic metabolic activity. Non-limiting examples of such killed vaccines include tetanus toxoid, diphtheria toxoid, and the killed whole cell pertussis vaccine. Other non-limiting examples of conventional non-living immunogens are haptens, anti-idiotype antibodies, and nucleic acid molecules, such as DNA or RNA, that can be expressed in cells as immunogenic molecules encoded by such nucleic acids. Alternatively, conventional immunogens may be classified according to their functional or structural properties in a microorganism such as capsular, fimbriae, nuclear, cell wall, membrane, and cytoplasmic immunogens.

Chemically speaking, immunogens of biological origin are most often peptides (including proteins), carbohydrates, glycopeptides, lipids, glycolipids, or lipopeptides.

Immunogens of the present invention may be pediatric or non-pediatric immunogens. The term “pediatric immunogens” refers to immunogens that after birth were routinely administered in 2004 to children less than one year old, in modern developed nations of moderate latitudes. These agents include but are not limited to BCG, measles, mumps, rubella, diphtheria, pertussis, hemophilus influenza, tetanus, hepatitis A, hepatitis B, varicella, influenza, pneumococcus, meningococcus and polio. Other immunogens are considered non-pediatric immunogens, and may include, but are not limited to, the group consisting of anthrax, plague, encephalitis, Hepatitis C, hepatitis e, typhus, typhoid fever, streptococcus, staphylococcus, lyme disease, cholera, campylobacter, helicobacter, E. coli, shigella, leishmania, leprosy, cytomegalovirus (CMV), human papilloma virus, respiratory syncytial virus, Epstein Barr virus, herpes, parainfluenza, rotavirus, adenovirus, human immunodeficiency virus (HIV), hepatitis A, NonA NonB hepatitis, rabies, yellow fever, rabies, Japanese encephalitis, flavivirus, dengue, west nile virus, avian flu virus, SARS coronaviruses, toxoplasmosis, coccidiomycosis, schistosomiasis,

The term immunogen used here is not intended to cover foods customarily given for nutritional reasons to infants and other children, such as bovine milk, common baby formula, and common baby food, even though such foods may nominally contain “immunogens”. It should be noted that researchers are developing edible vaccines comprising genetically engineered food protects that also protect against traditional infections such as polio. In such a case, the edible vaccine components would be considered immunogens.

Immunogens are distinct from immune modulators. There are several classes of immune modulators. One class is “immunocyte receptor ligands.” Members of this class of agents bind to cell receptors of immune mediator cells in a non-antigen specific manner to cause the induction of an immune response, e.g., as defined herein. One subclass of this group is cytokines. Cytokines that are produced by lymphocytes are termed lymphokines, whereas peptides produced by monocytes or macrophages are given the term monokines. Thus, the terms cytokines, lymphokines, and interleukins may be used interchangeably to designate those peptide molecules that modulate host responses to foreign antigens or host injury by regulating the growth, mobility and differentiation of leukocytes and other cells.

### Immunogenic Agents

An immunogenic agent (vaccine, immunogenic formulation) of the present invention is a pharmaceutically-acceptable composition comprising at least one immunogen in an amount such that, when administered according to an immunization schedule as disclosed above, it contributes to the desired effect against a chronic immune-mediated disorder, and also against an infectious disease.

When multiple immunogens, and/or multiple dosings of the same immunogen, are administered, the individual doses of individual immunogens may by themselves be subimmunogenic, provided that in aggregate, when administered according to the schedule, an immunogenic effect is achieved.

A hepatitis B immunogen may be recombinant or produced from blood products. A polio immunogen may be a live and or killed immunogen. A polio vaccine may be trivalent such that the vaccine may induce in mammals antibodies reactive to three serotypes of polio virus. A hemophilus influenza immunogen may be a conjugated and or unconjugated immunogen. A pertussis immunogen may be a non-whole cell or a whole cell immunogen. The non-whole cell immunogen may include a cellular pertussis immunogen.

In addition to the immunogen, the pharmaceutical composition may contain suitable pharmaceutically acceptable carriers, such as excipients, carriers and/or auxiliaries which facilitate processing of the active compounds into preparations which can be used pharmaceutically.

Such carriers may include depot adjuvants that release an immunogen in vivo over a prolonged period as compared to administration of an unbound immunogen. Preferably the depot adjuvant comprises an aluminum, calcium or salts thereof, such as aluminum sulfate (alum), aluminum phosphate, calcium phosphate or aluminum hydroxide.

Pharmaceutical compositions comprising at least one immunogen useful according to the present invention may also include suitable solutions for administration, intramuscularly, intravenously, subcutaneously, dermally, orally, mucosally, or rectally or by any other injection, and contain from about 0.001 to 99.999 percent, preferably from about 20 to 75 percent of active component (i.e. the immunogen) together with the excipient. Compositions which can be administered rectally include suppositories. Preparations of immunogenic agents for parenteral administration include sterile aqueous or non-aqueous solutions, suspensions, and emulsions, which may contain auxiliary agents or excipients, such as suitable adjuvants, which are known in the art. Pharmaceutical compositions such as tablets and capsules can also be prepared according to routine methods. See, e.g., Berker, infra, Goodman, infra, and Avery, infra, which are entirely incorporated herein by reference, including all references cited therein.

The immunogenic agents of the present invention may optionally include immunomodulators other than immunogens. Such immunomodulators may also be administered separately as a part of the program.

The compositions of the present invention may also include pharmaceuticals whose primary activity is non-immunological.

It is understood that the dosage of an immunogenic agent of the present invention administered in vivo or in vitro will be dependent upon the age, sex, health, and weight of the recipient, kind of concurrent treatment, if any, frequency of treatment, and the nature of the effect desired. The ranges of effective doses provided below are not intended to limit the invention and represent preferred dose ranges. However, the most preferred dosage will be tailored to the individual subject, as is understood and determinable by one of skill in the art, without undue experimentation. In the context of the present invention “one dose” may include concurrent or separate administration of more than one immunogen comprised of an immunogenic agent according to the present invention. See, e.g., Berkow et al, eds., The Merck Manual, 16th edition, Merck and Co., Rahway, N.J., 1992; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. 1987), which references and references cited therein, are entirely incorporated herein by reference.

The total dose, as in a pharmaceutically acceptable dose, required for each treatment may be administered by multiple doses or in a single dose. An immunogenic agent may be administered alone or in conjunction with other therapeutics directed to immunologic disorders, such as allergies, immune mediated cancers and autoimmune pathologies, as known in the art.

The pharmaceutically acceptable dosage of the immunogene will usually be about 0.01 ·mu·g to about 5 mg of immunogen, per kg body weight, and preferably from about 0.1 ·mu·g/kg to about 1 mg/kg body weight, still more preferably about 1 ·mu·g/kg to about 300 ·mu·g/kg, most preferably about 10 ·mu·g/kg to about 100 ·mu·g/kg. Nevertheless, Applicants' invention is not limited to the dosages set forth above. The active agent is the at least one immunogenic agent that induces an immune response according to the present invention. The safe dose will vary depending on the agent. Some immunogens are toxic at low doses while others are not.

### Pharmaceutical Purpose

The immunogenic agents of the present invention, whether or not incorporated into kits, may be pharmaceutically administered, according to an immunization schedule, to achieve a pharmaceutical purpose.

One pharmaceutical purpose of the invention is to protect subjects against at least one infectious disease, by administering one or more immunogens which elicit a protective immune response against such disease.

The term “protection”, as used herein, encompasses “prevention,” “suppression” or “treatment.” “Prevention” involves administration of a pharmaceutical composition prior to the induction of the disease. “Suppression” involves administration of the composition prior to the clinical appearance of the disease. “Treatment” involves administration of the protective composition after the appearance of the disease. Treatment may be ameliorative or curative.

It will be understood that in human and veterinary medicine, it is not always possible to distinguish between “preventing” and “suppressing” since the ultimate inductive event or events may be unknown, latent, or the patient is not ascertained until well after the occurrence of the event or events. Therefore, it is common to use the term “prophylaxis” as distinct from “treatment” to encompass both “preventing” and “suppressing” as defined herein. The term “protection,” as used herein, is meant to include “prophylaxis.”

The “protection” provided need not be absolute, i.e., the disease need not be totally prevented or eradicated, provided that there is a clinically recognizable improvement, preferably of a statistically significant character (p=0.05,) relative to a control population. Protection may be limited to mitigating the severity, rapidity of onset or duration of symptoms of the disease. An agent which provides protection to a lesser degree than do competitive agents may still be of value if the other agents are ineffective for a particular individual, if it can be used in combination with other agents to enhance the level of protection, or if it is safer than competitive agents.

The effectiveness of a treatment can be determined by comparing the duration, severity, etc. of the disease post-treatment with that in an untreated control group, preferably matched in terms of the disease stage.

The effectiveness of a prophylaxis will normally be ascertained by comparing the incidence of the disease in the treatment group with the incidence of the disease in a control group, where the treatment and control groups were considered to be of equal risk, or where a correction has been made for expected differences in risk.

In general, prophylaxis will be rendered to those considered to be at higher risk for the disease by virtue of family history, prior personal medical history, or elevated exposure to the causative agent.

The second pharmaceutical purpose of the invention is in prophylaxis against at least one chronic immune-mediated disorder. To the extent that administration of the vaccine is a risk factor for developing a CIMD, the first and second purposes are at odds. In this situation, the invention attempts to balance the risks of developing the infectious disease and developing the CIMD. That is, the invention attempts to achieve a clinically acceptable level of protection against the infectious disease while at the same time achieve a reduced risk of developing a CIMD, at least relative to the risk with an alternative immunization schedule.

### Pharmaceutical Administration

The immunogenic agents of the present invention may be administered by any effective route, for example, by various parenteral routes such as subcutaneous, intravenous, intradermal, intramuscular, intraperitoneal, intranasal, transdermal, or buccal routes. Alternatively, or concurrently, administration may be by the oral route. A preferred mode of using an immunogenic agent or composition of the present invention is by intramuscular application.

Having now generally described the invention, the same will be more readily understood through reference to the following examples which are provided by way of illustration, and not intended to be limiting of the present invention.

### EXAMPLES

A cohort analysis was performed in all children born in Denmark from Jan. 1, 1990 to Dec. 31, 2000. Children were followed for the development of type 1 diabetes through the age of 12 or until Dec. 31, 2001. The immunization schedules of each child was extracted from a computerized registry as was the development of diabetes. Cohorts were constructed based on what vaccines and how many doses the children received. Raw data was obtained from a published source (Hviid et al., 2004). The rate of diabetes was calculated for each cohort and the rate ratio was calculated by comparing the cohorts receiving the vaccines to an cohort that did not receive the vaccine (Table 1A). A separate but identical analysis was performed in the subgroup of children which had a family history of diabetes. The results are found in Table 1B. The results show that in the general population there was an elevated risk ratio associated with many different vaccines including the hemophilus, MMR, polio, whole cell pertussis, and the combined diphtheria, tetanus inactive polio vaccine. There were only a small number of children with a family history of type 1 diabetes this significantly reduces the power of the study. Still the results show statistically significant effects with the oral polio and the whole cell pertussis vaccine. In no case was there a decrease in the risk ratio in the group with a family history versus the general population when looking at the risk of receiving at least one dose of a vaccine. This indicates that there is no evidence that the risk ratio is not at least equally elevated in the group with a family history as it is in the general population. In general the relative risks were similar in the general population and the children with a family history of diabetes. The rate ratio associated with a single dose of pertussis vaccine in the group with the family history of diabetes (8.26) exceeds the confidence interval of the same group in the general population (0.93<<RR<<1.79). Likewise the rate ratio associated with 3 doses of the polio vaccine in the diabetic group (4.71) exceeds the upper limit of the confidence interval in the group in the general population (2.06<<RR<<3.08). By contrast the differences in the attributable or absolute risk were quite different between those in the general population and those in the diabetic group. For example in the group which received three doses of polio vaccine the attributable risk in the general population was 12 cases/100,000/year (20.86-8.27) while the attributable risk in the diabetic population was 720 cases/100,000/year (914-194).

The results significantly differ from a previous analysis (Hviid et al., 2004) of this data where only the “adjusted” results were compared. The previous analysis inappropriately adjusted for “age, sex and calendar period” and lead to flawed conclusions. Those conclusions include that vaccines were not associated with an increased risk of diabetes and those with a family history of diabetes were not at an increased risk of diabetes.

TABLE 1A
Diabetes After Immunization, All Children

| | | | Risk of diabetes associated with immunization | | | |
|---|---|---|---|---|---|---|
| | | | Absolut | Attrib | | |
| | | | Risk | Risk | | |
| | | | rate/ | rate/ | Relative Risk | |
| | person-years | No. of | 100,000/ | 100,000/ | rate | 95% confidence |
| Vaccine | in group | Cases | year | year | ratio | Interval |

Hemophilus Influenza

| | | | | | | |
|---|---|---|---|---|---|---|
| unvaccinated | 1,596,918 | 211 | 13.21 | | 1 | |
| 1 Dose | 835,833 | 123 | 14.72 | 1.50 | 1.11 | (0.89<<RR<<1.39) |
| 2 Doses | 850,946 | 114 | 13.40 | 0.18 | 1.01 | (0.81<<RR<<1.27) |
| 3 Doses | 1,436,820 | 233 | 16.22 | 3.00 | 1.23 | (1.02<<RR<<1.48) |
| At least 1 dose | 3,123,599 | 470 | 15.05 | 1.83 | 1.14 | (0.97<<RR<<1.34) |

Diphtheria, tetanus and inactivated polio

| | | | | | | |
|---|---|---|---|---|---|---|
| Unvaccinated | 1,110,803 | 110 | 9.90 | | 1 | |
| 1 Dose | 276,557 | 33 | 11.93 | 2.03 | 1.21 | (0.82<<RR<<1.78) |
| 2 Doses | 1,134,823 | 178 | 15.69 | 5.78 | 1.58 | (1.25<<RR<<2.01) |
| 3 Doses | 2,198,334 | 360 | 16.38 | 6.47 | 1.65 | (1.34<<RR<<2.05) |
| at least one dose | 3,609,714 | 571 | 15.82 | 5.92 | 1.60 | (1.60<<RR<<1.96 |

Diphtheria, tetanus, acellular pertussis and inactivated polio

| | | | | | | |
|---|---|---|---|---|---|---|
| Unvaccinated | 3,734,846 | 552 | 14.78 | | 1 | |
| 1 Dose | 296,026 | 39 | 13.17 | −1.61 | 0.89 | (0.64<<RR<<1.23) |
| 2 Doses | 242,792 | 24 | 9.89 | −4.89 | 0.67 | (0.44<<RR<<1.01) |
| 3 Doses | 446,854 | 66 | 14.77 | −0.01 | 1.00 | (0.77<<RR<<1.29) |
| at least one dose | 985,672 | 129 | 13.09 | −1.69 | 0.89 | (0.79<<RR<<1.07) |

Whole-cell pertussis

| | | | | | | |
|---|---|---|---|---|---|---|
| Unvaccinated | 995,949 | 109 | 10.94 | | 1 | |
| 1 Dose | 382,317 | 54 | 14.12 | 3.18 | 1.29 | (0.93<<RR<<1.79) |
| 2 Doses | 1,383,584 | 194 | 14.02 | 3.08 | 1.28 | (1.01<<RR<<1.62) |
| 3 Doses | 1,958,668 | 324 | 16.54 | 5.60 | 1.51 | (1.22<<RR<<1.88) |
| at least one dose | 3,724,569 | 572 | 15.36 | 4.41 | 1.40 | (1.14<<RR<<1.72) |

Measle Mumps Rubella

| | | | | | | |
|---|---|---|---|---|---|---|
| unvaccinated | 1,373,401 | 124 | 9.03 | | 1 | |
| 1 dose | 2,934,287 | 499 | 17.01 | 7.98 | 1.88 | (1.55<<RR<<2.29) |
| unknown | 412,830 | 58 | 14.05 | | | |

Oral Polio

| | | | | | | |
|---|---|---|---|---|---|---|
| Unvaccinated | 1,655,931 | 137 | 8.27 | | 1 | |
| 1 Dose | 742,807 | 95 | 12.79 | 4.52 | 1.55 | (1.19<<RR<<2.01) |
| 2 Doses | 825,780 | 137 | 16.59 | 8.32 | 2.01 | (1.58<<RR<<2.54) |
| 3 Doses | 1,496,000 | 312 | 20.86 | 12.58 | 2.52 | (2.06<<RR<<3.08) |
| at least one dose | 3,064,587 | 544 | 17.75 | 9.48 | 2.15 | (1.78<<RR<<2.59) |

TABLE 1B
Diabetes After Immunization, Children with family history of diabetes

| | | | Risk of diabetes associated with immunization | | | |
|---|---|---|---|---|---|---|
| | | | Absolut | Attrib | | |
| | | | Risk | Risk | | |
| | | | rate/ | rate/ | Relative Risk | |
| | person-years | No. of | 100,000 | 100,000 | Rate | 95% CONFIDENCE |
| Vaccine | in group | Cases | per year | per year | Ratio | INTERVAL |

Hemophilus Influenza

| | | | | | | |
|---|---|---|---|---|---|---|
| unvaccinated | 1419 | 7 | 493 | | | |
| 1 Dose | 799 | 7 | 876 | 383 | 1.78 | (0.63<<RR<<5.05) |
| 2 Doses | 709 | 2 | 282 | −211) | 0.57 | (0.12<<RR<<2.75) |
| 3 Doses | 1281 | 10 | 781 | 287 | 1.58 | (0.60<<RR<<4.15) |
| At least 1 dose | 2789 | 19 | 681 | 188 | 1.38 | (0.58<<RR<<3.28) |

Diphtheria, tetanus and inactivated polio

| | | | | | | |
|---|---|---|---|---|---|---|
| Unvaccinated | 258 | 1 | 388 | | | |
| 1 Dose | 1092 | 8 | 733 | 345 | 1.89 | (0.24<<RR<<15.05) |
| 2 Doses | 2136 | 16 | 749 | 361 | 1.93 | (0.26<<RR<<14.51) |
| 3 Doses | 723 | 1 | 138 | −249) | 0.36 | (0.02<<RR<<5.68) |
| at least one dose | 3951 | 25 | 633 | 245 | 1.63 | (0.22<<RR<<12.0) |

Diphtheria, tetanus, acellular pertussis and inactivated polio

| | | | | | | |
|---|---|---|---|---|---|---|
| Unvaccinated | 3437 | 21 | 611 | | | |
| 1 Dose | 258 | 4 | 1,550 | 939 | 2.54 | (0.88<<RR<<7.34) |
| 2 Doses | 167 | 0 | 0 | −611) | 0.00 | (0.00<RR<<4.91) |
| 3 Doses | 347 | 1 | 288 | −323) | 0.47 | (0.06<<RR<<3.50) |
| at least one dose | 772 | 5 | 648 | 37 | 1.06 | (0.40<<RR<<2.80) |

Whole-cell pertussis

| | | | | | | |
|---|---|---|---|---|---|---|
| Unvaccinated | 721 | 2 | 277 | | | |
| 1 Dose | 349 | 8 | 2,292 | 2,015 | 8.26 | (1.76<<RR<<38.71) |
| 2 Doses | 1298 | 14 | 1,079 | 801 | 3.89 | (0.89<<RR<<17.06) |
| 3 Doses | 1841 | 2 | 109 | −169) | 0.39 | (0.06<<RR<<2.78) |
| at least one dose | 3488 | 24 | 688 | 411 | 2.48 | (0.59<<RR<<10.47) |

Measle Mumps Rubella

| | | | | | | |
|---|---|---|---|---|---|---|
| unvaccinated | 1053 | 6 | 570 | | | |
| 1 dose | 2795 | 20 | 716 | 146 | 1.26 | (0.51<<RR<<3.12) |
| unknown | 361 | 0 | 0 | | | |

Oral Polio

| | | | | | | |
|---|---|---|---|---|---|---|
| Unvaccinated | 1030 | 2 | 194 | | | |
| 1 Dose | 591 | 3 | 508 | 313 | 2.61 | (0.44<<RR<<15.60) |
| 2 Doses | 837 | 5 | 597 | 403 | 3.08 | (0.60<<RR<<15.82) |
| 3 Doses | 1750 | 16 | 914 | 720 | 4.71 | (1.08<<RR<<20.44) |
| at least one dose | 3178 | 24 | 755 | 561 | 3.89 | (0.92<<RR<<16.43) |

### REFERENCES

1. Graves P M, Barriga K J, Norris J M, et al. Lack of Association Between Early Childhood Immunizations and -Cell Autoimmunity. Diabetes Care 1999; 22:1694.
2. Hummel M, Schenker M, Fuchtenbusch M, Ziegler AG. No major association of breast feeding, vaccinations, and childhood viral diseases with early islet autoimmunity in the German BABYDIAB study. Diabetes Care 2000; 23:969-74.
3. Hviid A, Stellfeld M, Wohlfahrt J, Melbye M. Childhood vaccination and type 1 diabetes. NEJM 2004; 350:1398-404.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference. Reference to steps or compositions as being “conventional”, “standard”, “usual”, “known” or the like is not to be considered an admission that any aspect, description or embodiment of the present invention is disclosed, taught or suggested in the relevant art.

Any description of an embodiment as “desirable” or “preferable” is intended to imply that the invention is not limited to the stated embodiment, but rather covers alternatives whether mentioned or not. Any description of a class or range as being useful or preferred in the practice of the invention shall be deemed a description of any subclass or subrange contained therein, as well as a separate description of each individual member or value in said class or range.

1. A method for more safely marketing one or more doses of one or more immunogens which are protective against one or more infectious diseases when administered according to one or more immunization schedules, said method comprising providing information, or assisting in the dissemination of information, on the risk of developing at least one chronic immune mediated disorder, in a human who has received one or more doses of at least one immunogen according to at least one immunization schedule, wherein said human was, prior to said receipt, at an increased risk, unrelated to or irrespective of prior immunization, sex, race or nationality for developing said chronic immune mediated disorder, and providing one or more of said immunogens for use in one or more immunization schedules, said information subsequently being used in the consideration of the association between one or more doses of at least one immunogen in an immunization schedule and the risk of one or more chronic immune mediated disorders in a human who is already at an increased risk, unrelated to or irrespective of prior immunization, sex, race or nationality, for developing a chronic immune mediated disorder, wherein the risk considered for an association with at least one immunogen comprises:
a) the attributable risk or absolute risk, and/or
b) the relative risk (risk ratio), wherein said relative risk is not adjusted for calendar period, and/or
c) odds ratio
said risk subsequently being considered during the selection of a human subject and of an immunization schedule, employing at least one of said provided immunogens, and protective against at least one infectious disease, for immunization of said selected human subject, said selected human subject subsequently being immunized against one or more infectious diseases according to said selected immunization schedule.

2. The method of claim 1 wherein said human is at an increased risk for developing a chronic immune mediated disorder based on family history.

3. The method of claim 2 wherein said chronic immune mediated disorder comprises diabetes.

4. The method of claim 1 wherein said chronic immune mediated disorder comprises diabetes.

5. The method of claim 1 wherein two or more immunization schedules are compared and wherein said two or more immunization schedules contain at least one immunogen in common and the schedules differ by the formulation and or the number of doses administered and or the timing of administration of the common immunogen.

6. The method of claim 5 wherein said human is at increased risk for developing a chronic immune mediated disorder based on family history.

7. The method of claim 6 wherein said disorder wherein at least one of said disorders is” diabetes.

8. (canceled)

9. The method of claim 1 wherein condition a applies.

10. (canceled)

11. The method of claim 2 wherein condition a applies.

12-13. (canceled)

14. The method of claim 2 wherein at least one of said chronic immune mediated disorders is neither a neurological disorder nor diabetes.

15-18. (canceled)

19. The method of claim 3 wherein condition b applies and the risk of condition b is statistically significant.

20-41. (canceled)

42. The method of claim 2 wherein at least one immunogen other than a hepatitis B immunogen is provided.

43. The method of claim 3 wherein at least one immunogen other than a hepatitis B immunogen is provided.

44-51. (canceled)

52. The method of claim 42 wherein at least one immunogen is a hemophilus B immunogen.

53. The method of claim 43 wherein at least one immunogen is a hemophilus B immunogen.

54-61. (canceled)

62. The method of claim 42 wherein at least one immunogen is a measles, mumps or rubella immunogen.

63. The method of claim 43 wherein at least one immunogen is a measles, mumps or rubella immunogen.

64-71. (canceled)

72. The method of claim 42 wherein at least one immunogen is a polio immunogen.

73. The method of claim 43 wherein at least one immunogen is a polio immunogen.

74-81. (canceled)

82. The method of claim 42 wherein at least one immunogen is a pertussis immunogen.

83. The method of claim 43 wherein at least one immunogen is a pertussis immunogen.

84-98. (canceled)

98. The method of claim 1 wherein said data is from a cohort analysis, condition a applies and the absolute or attributable risk is greater than or equal to 250 cases/100,000.

99. The method of claim 2 wherein said data is from a cohort analysis, condition a applies and the absolute or attributable risk is greater than or equal to 250 cases/100,000.

100. The method of claim 3 wherein said data is from a cohort analysis, condition a applies and the absolute or attributable risk is greater than or equal to 250 cases/100,000.

101-138. (canceled)

139. A method for more safely administering one or more doses of one or more immunogens which protect a human against one or more infectious diseases when administered according to one or more immunization schedules, said method comprising
I. considering the association between the administration of one or more doses of at least one immunogen according to an immunization schedule, and the risk of one or more chronic immune mediated disorders in a human who is already at an increased risk, unrelated to or irrespective of prior immunization, sex, race or nationality, for developing a chronic immune mediated disorder wherein the risk considered for an association with at least one immunogen comprises:
a) the attributable risk or absolute risk, and/or
b) the relative risk (risk ratio), wherein said relative risk is not adjusted for calendar period, and/or,
c) the odds ratio,
II. selecting a particular human subject, and an immunization schedule expected to be protective against one or more infectious diseases, wherein such selection is at least in part reflective of the consideration set forth in I above, and
III. administering one or more doses of at least one immunogen, according to an immunization schedule, to said subject.

140-143. (canceled)

144. A method for more safely providing and administering one or more doses of one or more immunogens which are protective against one or more infectious diseases when administered according to one or more immunization schedules, said method comprising providing information, or assisting in the dissemination of information, on the risk of developing at least one chronic immune mediated disorder, in a human who has received one or more doses of at least one immunogen according to at least one immunization schedule, wherein said human was, prior to said receipt, at an increased risk, unrelated to or irrespective of prior immunization, sex, race or nationality for developing said chronic immune mediated disorder, providing one or more of said immunogens for use in one or more immunization schedules, considering the association between one or more doses of at least one immunogen in an immunization schedule and the risk of one or more chronic immune mediated disorders in a human who is already at an increased risk, unrelated to or irrespective of prior immunization, sex, race or nationality, for developing a chronic immune mediated disorder, wherein the risk considered for an association with at least one immunogen comprises:
a) the attributable risk or absolute risk, and/or
b) the relative risk (risk ratio), wherein said relative risk is not adjusted for calendar period, and/or
c) the odds ratio,
identifying a human subject to be immunized and/or selecting one or more doses of one or immunogens to be administered to a human subject wherein a decision on administering at least one immunogen or the number of doses of at least one immunogen or the scheduling of at least one immunogen is at least in part reflective of said considering step, and immunizing said human against said one or more infectious diseases.

145. A method for more safely marketing one or more doses of one or more immunogens which induce protective immunity in a human to one or more infectious diseases when administered according to one or more immunization schedules, said method comprising providing one or more immunogens for use in one or more immunization schedules after providing or assisting in the dissemination of information on the risk of one or more doses of at least one immunogen in said one or more immunization schedules in a human who is already at an increased risk, unrelated to or irrespective of prior immunization, sex, race or nationality for developing a chronic immune mediated disorder such that said one or more immunogens provided is administered according to an immunization method which comprises:
I. considering the association between one or more doses of at least one immunogen in an immunization schedule and the risk of one or more chronic immune mediated disorders in a human who is already at an increased risk, unrelated to or irrespective of prior immunization, sex, race or nationality, for developing a chronic immune mediated disorder Where the risk considered for an association with at least one immunogen is:
a) the absolute or attributable risk, and/or
b) the relative risk (risk ratio), wherein said relative risk is not adjusted for calendar period,
and/or
c) the odds ratio,
II. identifying a human subject to be immunized and or selecting one or more doses of one or immunogens to be administered to a human subject where a decision on administering at least one immunogen or the number of doses of at least one immunogen or the scheduling of at least one immunogen is in part reflective of the consideration in I, and
III. immunizing said human against said one or more infectious diseases.
